(19) United States
(12) Patent Application Publication
(10) Pub. No.: US 2017/0361112 A1
(43) Pub. Date: Dec. 21, 2017

(54) External Charger for an Implantable Medical Device Having a Multi-Layer Magnetic Shield

(71) Applicant: Boston Scientific Neuromodulation Corporation, Valencia, CA (US)

(72) Inventor: Thomas W. Stouffer, Chatsworth, CA (US)

(21) Appl. No.: 15/616,428

(22) Filed: Jun. 7, 2017

Related U.S. Application Data
(60) Provisional application No. 62/350,626, filed on Jun. 15, 2016.

(51) Int. Cl. A61N 1/378; H01F 38/14; H01F 27/28; A61N 1/36; A61N 1/372; H02J 50/12; H02J 7/02; H02J 50/80; A61N 1/05

(52) U.S. Cl. CPC A61N 1/3787; H02J 7/027; H02J 7/025; H01F 38/14; H01F 27/2885; H01F 27/2804; H02J 50/80; A61N 1/36142; A61N 1/36125; A61N 1/37229; H02J 50/12; A61N 1/0534; A61N 1/0551

(57) ABSTRACT

An external charger for an implantable medical device (IMD) includes a multi-layer shield to direct the magnetic field generated by its charging coil towards the IMD. Each of the shield's multiple layers includes a ferromagnetic material that increases the permeance of the magnetic field's flux paths. The layers decrease in magnetic saturation point with increasing distance from the external charger's charging coil. That is, the layer closest to the charging coil has a higher saturation point than the next layer further from the charging coil, and so on. Layers that are positioned closer to the charging coil shield layers that are further from the charging coil, which generally have higher magnetic permeabilities, such that the magnetic intensity does not exceed any layer's saturation point. In this way, the multi-layer shield provides a beneficial balance between permeability and saturation, which can limit the required dimensions of the shield.

### CROSS-REFERENCE TO RELATED APPLICATIONS

This is a non-provisional of U.S. Provisional Patent Application Ser. No. 62/350,626, filed Jun. 15, 2016, to which priority is claimed, and which is incorporated herein by reference in its entirety.

### FIELD OF THE INVENTION

The present invention relates to wireless external chargers for use in implantable medical device systems.

### BACKGROUND

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system, including a Deep Brain Stimulation (DBS) system.

As shown in FIGS. 1A-1C, a SCS system typically includes an Implantable Pulse Generator (IPG) 10 (Implantable Medical Device (IMD) 10 more generally), which includes a biocompatible device case 12 formed of a conductive material such as titanium for example. The case 12 typically holds the circuitry and battery 14 (FIG. 1C) necessary for the IMD 10 to function, although IMDs can also be powered via external RF energy and without a battery. The IMD 10 is coupled to electrodes 16 via one or more electrode leads 18, such that the electrodes 16 form an electrode array 20. The electrodes 16 are carried on a flexible body 22, which also houses the individual signal wires 24 coupled to each electrode. In the illustrated embodiment, there are eight electrodes (Ex) on each lead 18, although the number of leads and electrodes is application specific and therefore can vary. The leads 18 couple to the IMD 10 using lead connectors 26, which are fixed in a non-conductive header material 28, which can comprise an epoxy for example.

As shown in the cross-section of FIG. 1C, the IMD 10 typically includes a printed circuit board (PCB) 30, along with various electronic components 32 mounted to the PCB 30. Two coils (more generally, antennas) are shown in the IMD 10: a telemetry coil 34 used to transmit/receive data to/from an external controller (not shown); and a charging coil 36 for charging or recharging the IMD's battery 14 using an external charger, which is discussed in detail later.

FIG. 2 shows the IMD 10 in communication with an external charger 50 used to wirelessly convey power to the IMD 10, which power can be used to recharge the IMD's battery 14. The transfer of power from the external charger 50 is enabled by a primary charging coil 52. The external charger 50, like the IMD 10, also contains a PCB 54 on which electronic components 56 are placed. Some of these electronic components 56 are discussed subsequently. A user interface 58, including touchable buttons and perhaps a display and a speaker, allows a patient or clinician to operate the external charger 50. A battery 60 provides power for the external charger 50, which battery 60 may itself be rechargeable. The external charger 50 can also receive AC power from a wall plug. A hand-holdable housing 62 sized to fit a user's hand contains all of the components.

Power transmission from the external charger 50 to the IMD 10 occurs wirelessly and transcutaneously through a patient's tissue 25, via inductive coupling. FIG. 3 shows details of the circuitry used to implement such functionality. Primary charging coil 52 in the external charger 50 is energized via charging circuit 64 with an AC current, Icharge, to create an AC magnetic charging field 66. This magnetic field 66 induces a current in the secondary charging coil 36 within the IMD 10, providing a voltage across coil 36 that is rectified (38) to DC levels and used to recharge the battery 14, perhaps via battery charging and protection circuitry 40 as shown. The frequency of the magnetic field 66 can be perhaps 80 kHz or so. When charging the battery 14 in this manner, it is typical that the housing 62 of the external charger 50 touches the patient's tissue 25, perhaps with a charger holding device or the patient's clothing intervening, although this is not strictly necessary.

The IMD 10 can also communicate data back to the external charger 50 during charging using reflected impedance modulation, which is sometimes known in the art as Load Shift Keying (LSK). This involves modulating the impedance of the charging coil 36 with data bits (“LSK data”) provided by the IMD 10's control circuitry 42 to be serially transmitted from the IMD 10 to the external charger 50. For example, and depending on the logic state of a bit to be transmitted, the ends of the coil 36 can be selectively shorted to ground via transistors 44, or a transistor 46 in series with the coil 36 can be selectively open circuited, to modulate the coil 36's impedance. At the external charger 50, an LSK demodulator 68 determines whether a logic ‘0’ or ‘1’ has been transmitted by assessing the magnitude of AC voltage Vcoil that develops across the external charger's coil 52 in response to the charging current Icharge and the transmitted data, which data is then reported to the external charger's control circuitry 72 for analysis. Such back telemetry from the IMD 10 can provide useful data concerning charging to the external charger 50, such as the capacity of the IMD's battery 14, or whether charging of the battery 14 is complete and operation of the external charger 50 and the production of magnetic field 66 can cease. LSK communications are described further for example in U.S. Patent Application Publication 2013/0096652.

External charger 50 can also include one or more thermistors 71, which can be used to report the temperature (expressed as voltage Vtherm) of external charger 50 to its control circuitry 72, which can in turn control production of the magnetic field 66 such that the temperature remains within safe limits. See, e.g., U.S. Pat. No. 8,321,029, describing temperature control in an external charging device.

Vcoil across the external charger's charging coil 52 can also be assessed by alignment circuitry 70 to determine how well the external charger 50 is aligned relative to the IMD 10. Generally speaking, if the external charger 50 is well aligned with the IMD 10, then Vcoil will drop as the charging circuitry 64 provides the charging current Icharge to the charging coil 52. Accordingly, alignment circuitry 70 can compare Vcoil, preferably after it is rectified 76 to a DC voltage, to an alignment threshold, Vt. If Vcoil<Vt, then external charger 50 considers itself to be in good alignment with the underlying IMD 10. If Vcoil>Vt, then the external charger 50 will consider itself to be out of alignment, and can indicate that fact to the patient so that the patient can attempt to move the charger 50 into better alignment. For example, the user interface 58 of the charger 50 can include an alignment indicator 74. The alignment indicator 74 may comprise a speaker (not shown), which can “beep” at the patient when misalignment is detected. Alignment indicator 74 can also or alternatively include one or more Light Emitting Diodes (LED(s); not shown), which may similarly indicate misalignment.

Providing the user with some indication of alignment is important because if the external charger 50 is not well aligned to the IMD 10, the magnetic field 66 produced by the charging coil 52 will not efficiently be received by the charging coil 36 in the IMD 10. Efficiency in power transmission can be quantified as the “coupling” between the transmitting coil 52 and the receiving coil 36 (k, which ranges between 0 and 1), which generally speaking comprises the extent to which power expended at the transmitting coil 52 in the external charger 50 is received at the receiving coil 36 in the IMD 10. It is generally desired that the coupling between coils 52 and 36 be as high as possible: higher coupling results in faster charging of the IMD battery 14 with the least expenditure of power in the external charger 50. Poor coupling is disfavored, as this will require high power drain (e.g., a high Icharge) in the external charger 50 to adequately charge the IMD battery 14. The use of high power depletes the battery 60 in the external charger 50, and more importantly can cause the external charger 50 to heat up, and possibly burn or injure the patient.

The coupling between coils 52 and 36 is also improved through the use of a shield 80 that is positioned to focus the magnetic field 66 toward the coil 36. The shield 80 is constructed from a ferromagnetic material having a high magnetic permeability. Such materials can include iron, cobalt, nickel, manganese, chromium, as well as oxides, alloys, and other combinations of these metals for example. The shield 80 increases the coupling between the coils 52 and 36 in three ways. First, the magnetic permeability of the shield 80, being substantially higher than the magnetic permeability of air and other non-ferromagnetic materials, increases the permeance of magnetic flux paths generated as a result of the energization of the coil 52. For a given magnetomotive force (e.g., a given current through the fixed number of turns in the coil 52), magnetic flux is proportional to the permeance of the magnetic circuit. Thus, an increase in the permeance of the magnetic flux paths results in an increase in the magnetic flux through any cross-sectional area perpendicular to the paths, most importantly through the coil 36. Second, as shown in FIGS. 4 (which shows the flux paths with no shield) and 5 (which shows the flux paths with shield 80), the shield 80 alters the shape of the magnetic field 66 such that the length of the flux path is shortened. For a given magnetomotive force, magnetic flux is inversely proportional to the length of the flux path. Thus, decreasing the length of the flux path also increases the flux through the coil 36. Third, the shield 80 alters the shape of the magnetic field 66 such that the components of the charger 50 above the shield 80 are substantially unexposed to the field 66. Exposure of any conductive components (such as, perhaps, the components 56 or the battery 60) to the field 66 results in the generation of eddy currents that produce a magnetic field in an opposite direction of the field 66. By substantially reducing the generation of eddy currents that oppose the field 66, the shield 80 additionally increases the flux through the coil 36. Reducing the generation of eddy currents also has the beneficial effect of reducing the generation of heat in the charger 50. Because the shield 80 improves coupling between the coils 52 and 36, its use enables the charger 50 to operate at lower charging currents or for the coil 52 to be designed with a smaller number of turns (or some combination of both).

### BRIEF DESCRIPTION OF THE DRAWINGS

FIGS. 1A-1C show different views of an implantable pulse generator, a type of implantable medical device (IMD), in accordance with the prior art.

FIG. 2 shows an external charger being used to charge a battery in an IMD, in accordance with the prior art.

FIG. 3 shows circuitry in an external charger and an IMD, in accordance with the prior art.

FIG. 4 shows the magnetic flux paths created by an external charger that does not include a shield, in accordance with the prior art.

FIG. 5 shows the magnetic flux paths created by an external charger that includes a shield, in accordance with the prior art.

FIG. 6 includes charts that show the relationship of magnetic permeability and coil inductance as a function of charging current, in accordance with the use of a prior art shield.

FIG. 7 shows an external charger that includes a multi-layer shield, in accordance with an example of the invention.

FIG. 8 shows the layers of a modified shield, in accordance with an example of the invention.

FIG. 9 is a chart that shows the magnetic permeabilities of the layers of a multi-layer shield as a function of magnetic field intensity, in accordance with an example of the invention.

FIG. 10 shows the magnetic flux paths created by an external charger that includes a multi-layer shield, in accordance with an example of the invention.

FIG. 11 is a chart that illustrates the magnetic flux through shields of varying types, in accordance with an example of the invention.

FIG. 12 is a chart that shows the inductance of an external charger's charging coil as a function of charging current for shields of varying types, in accordance with an example of the invention.

FIGS. 13A and 13B show different views of a charging system that incorporates a multi-layer shield, in accordance with an example of the invention.

FIG. 14 shows circuitry of the charging system illustrated in FIGS. 13A and 13B, in accordance with an example of the invention.

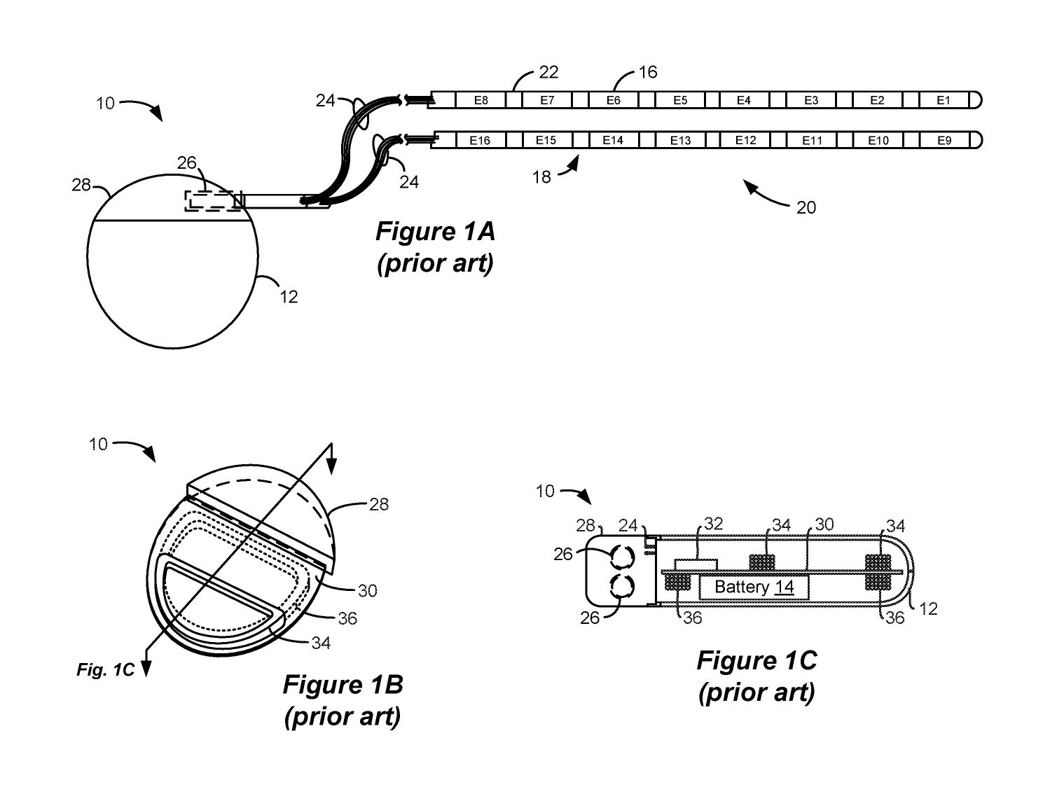

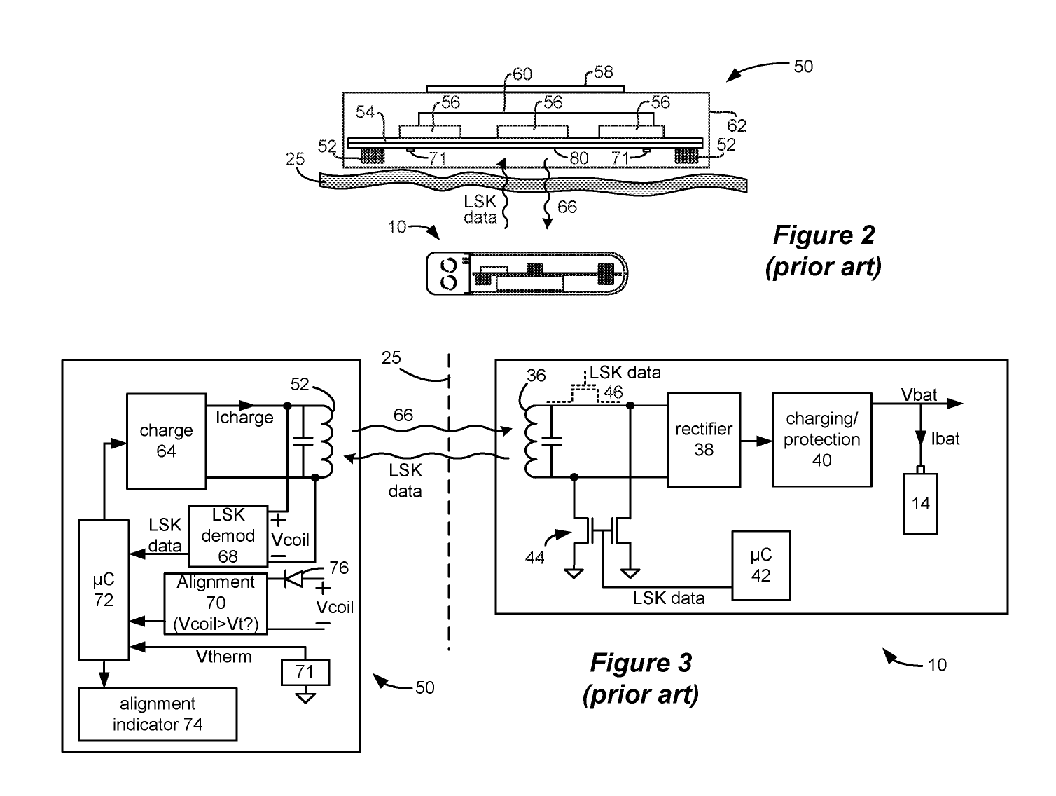

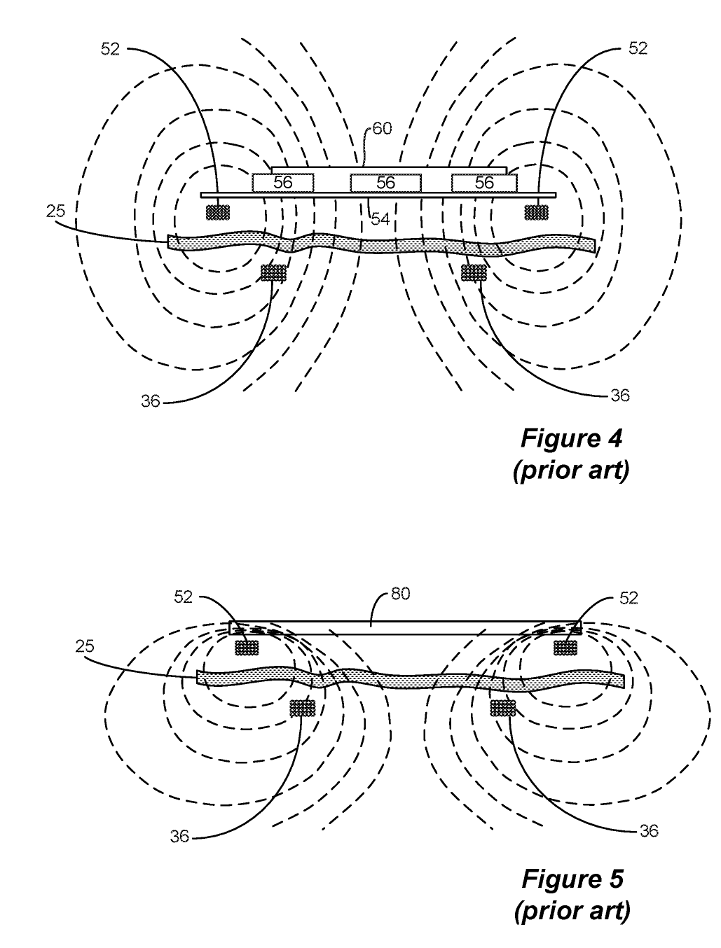

### DETAILED DESCRIPTION

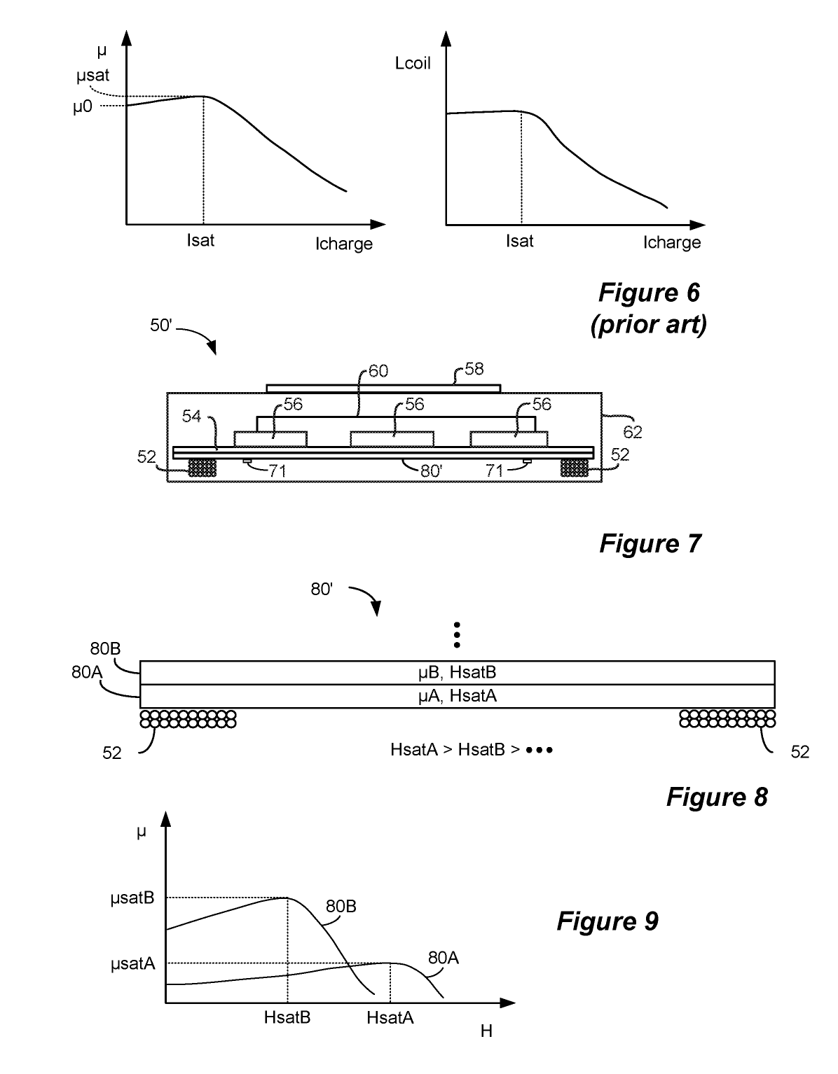

While the shield 80 improves the coupling between the coils 52 and 36, its use in the charger 50 also creates certain challenges. As is known, the magnetic permeability of ferromagnetic materials such as those from which the shield 80 is constructed varies as a function of the intensity H of the magnetic field (e.g., field 66) of a flux path through the material. Thus, the magnetic permeability of the shield 80 varies with the charging current Icharge. As shown in the chart in FIG. 6, the magnetic permeability of the material increases from its vacuum permeability (μ0) when no current is flowing through the coil (H=0) to a maximum permeability (μsat) at the material's “saturation point” before dropping sharply as the magnetic field intensity further increases. As also shown in FIG. 6, the sharp decrease in permeability of the shield results in a corresponding sharp decrease in the inductance Lcoil of coil 52 at charging currents above the saturation current Isat. This problem is exacerbated by the fact that materials having the highest magnetic permeability (i.e., the materials that most significantly increase coupling between the coils 52 and 36) generally saturate at a lower magnetic field intensity. Thus, a material having a more desirable (i.e., higher) magnetic permeability generally saturates at a lower charging current and a material having a more desirable (i.e., higher) saturation point generally has a lower magnetic permeability.

The inventor has recognized that it would be beneficial for the charger's control circuitry 72 to control the magnitude of Icharge to obtain a desired charging rate of the IMD 10's battery 14 and to control the frequency of Icharge so that the coil 52 operates at its resonant frequency. These values of the magnitude and frequency of Icharge are affected by the orientation of the charger 50 with respect to the IMD 10 due to changes in the mutual inductance between the coils 52 and 36. The inventor has also recognized that while the control circuitry 72 is capable of adjusting the frequency of the charging current to correct gradual changes in the resonant frequency, the change in the resonant frequency caused by the sharp decrease in Lcoil as a result of the magnetic saturation of the shield 80 is extremely difficult to control and often requires a “reset” of the charge control scheme whereby the charging current is substantially reduced and then gradually increased back to desired levels. Such control “resets” are time-consuming and inefficient as they can substantially increase the amount of time that is required to charge the battery 14.

There are a few ways in which this problem can be avoided, but each has its own drawback. The thickness of the shield 80 can be increased to increase the value of Isat, but that undesirably increases the size and weight of the charger. The maximum value of Icharge can be limited so that it cannot exceed Isat, but that limits the rate at which the IMD 10 can be recharged, especially for non-ideal orientations of the charger 50 relative to the IMD 10. The shield 80 can be eliminated altogether, but that forgoes the beneficial effects that the shield provides.

The inventor has conceived of a modified shield 80′ that strikes a balance between magnetic permeability and saturation and beneficially directs the magnetic field 66 towards the IMD 10. FIG. 7 illustrates a modified charger 50′ that includes a multi-layer shield 80′. The shield 80′ may be held in its position between the coil 52 and the PCB 54 by adhering the shield 80′ to the PCB 54 (i.e., affixing the shield 80′ to the PCB 54 with an adhesive) or by mechanically affixing the shield 80′ (e.g., to the case 62 or other component of the charger 50′) using a clamp or similar support structure (not shown). While the shield 80′ is illustrated as being positioned between the coil 52 and the PCB 54, this positioning is not strictly necessary and the shield 80′ may be positioned at other locations so long as the charging coil 52 is positioned between the shield 80′ and the external surface of the charger that is configured to be placed towards the patient's tissue. For example, the shield 80′ could be mounted against the top portion of the case 62 directly opposite the user interface 58. Other than the replacement of the shield 80 with the modified shield 80′, the other components and circuitry of the modified charger 50′ are unchanged from the charger 50.

As shown in FIG. 8, the shield 80′ includes multiple ferromagnetic layers (i.e., layers that include ferromagnetic materials) that saturate at different magnetic field intensities. While two layers 80A and 80B are shown, the shield 80′ can employ a greater number of layers. In one embodiment, each layer may be approximately 1-2 mm thick. The layers 80A and 80B are illustrated as the same thickness, but this is not strictly necessary and the thicknesses of the layers can be either the same or different. In a preferred embodiment, the shield 80′ has the same general shape as the coil 52. For example, the shield 80′ and the coil 52 may both be generally circular, generally rectangular, generally square, or some other shape, but the shield 80′ can also take a different shape than the coil. Further, the shield 80′ and coil 52 may be concentric (i.e., share the same center), although this is also not necessary. The shield 80′ may also be generally the same size as the coil 52, although it can also be larger or smaller than the coil 52. In one embodiment, each layer may be cut to the desired size and shape from a sheet of ferromagnetic material. For example, each layer may be cut to size using a die cutting machine. In one embodiment, the layers of the shield 80′ are affixed to each other by an adhesive applied between the layers.

While the layers 80A and 80B can be constructed from any ferromagnetic material, in a preferred embodiment one or more of the layers may comprise a ferrite material. Such ferrite materials are generally rigid, and a rigid ferrite material having the desired dimensions (i.e., relatively large compared to thickness) may be relatively brittle and subject to cracking. While the layers 80A and 80B can be formed from a ferrite material in this rigid form, because a significant crack in any layer can substantially reduce the effectiveness of the shield 80′, in a preferred embodiment, the ferrite materials for one or more layers may be pre-scored (e.g., to create a grid of small squares) and held together by a component such as a polymer film, for example. These types of pre-scored materials are more flexible and less susceptible to cracking. Examples of the types of ferrite materials that can form the various layers can include manganese-zinc ferrite, nickel-zinc ferrite, strontium ferrite, barium ferrite, and cobalt ferrite. While the ferromagnetic materials of the various layers may be positioned in direct contact with each other, the layers may also be separated by a thin barrier, such as the film that holds a pre-scored ferrite sheet together.

The layers are arranged such that the layer closest to the coil 52 saturates at the highest magnetic field intensity (i.e., has the highest magnetic saturation point). That is, the saturation point of the first layer 80A occurs at a higher magnetic field intensity (and thus a higher Icharge) than the saturation point of the second layer 80B (i.e., HsatA>HsatB), and so on for any additional layers. As described above, while not an absolute law, materials that saturate at a higher magnetic field intensity generally have a lower magnetic permeability. Therefore, the above saturation point relationship of the layers (i.e., HsatA>HsatB) generally corresponds to the opposite magnetic permeability relationship (i.e., μA<μB). This general relationship is illustrated in FIG. 9, which shows example magnetic permeabilities of the layers 80A and 80B as a function of magnetic field intensity. The shield 80′ only makes sense where the general relationship between saturation and magnetic permeability applies. This is true because if the layer 80A, which saturates at a higher magnetic field intensity than the layer 80B, is also superior to layer 80B in terms of magnetic permeability, then nothing is gained by adding the layer 80B. Instead, a single layer shield 80 constructed of the material of the first layer 80A would be superior to a multi-layer shield. However, as shown below, where the general relationship between magnetic permeability and saturation applies, a multi-layer shield 80′ can provide a beneficial balance between permeability and saturation.

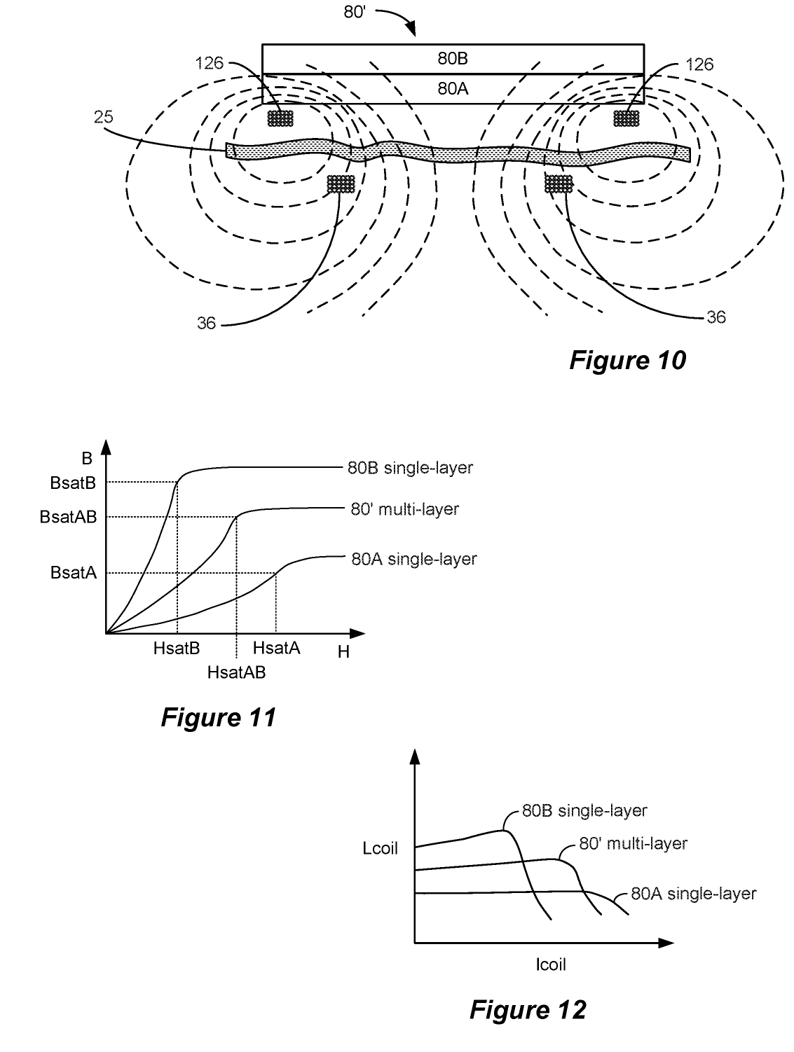

As illustrated in FIG. 10, the flux paths having the highest magnetic intensity H (i.e., those that have the shortest path around the coil 52) flow through the first layer 80A in the multi-layer shield 80′. The magnetic permeability of the layer 80A acts to increase the flux through the coil 36 in the same way as the single layer shield 80 described above. The layer 80A also acts to shield the layer 80B such that the flux paths flowing through the layer 80B are at an intensity level H that is below the saturation point of layer 80B. As a result, the multi-layer shield 80′ also takes advantage of the even higher magnetic permeability of the layer 80B in a manner that avoids the risk of layer 80B exceeding its saturation point. As will be understood, the number of layers and the thicknesses of the various layers can be selected to obtain the desired flux through the layers over a range of operating conditions (i.e., a range of Icharge values). For example, the thicknesses of the layers can be adjusted such that at the maximum magnetic intensity that can be generated by the coil (i.e., the maximum Icharge), the flux paths that flow through a layer closer to the coil 52 (e.g., layer 80A) and into a layer further from the coil 52 (e.g., layer 80B) have a magnetic intensity that is just below the saturation point of the layer further from the coil 52.

The balance between magnetic permeability and saturation that is achieved by the shield 80′ is illustrated in FIG. 11, which shows example plots of the magnetic flux B through a single layer shield formed entirely of the material of layer 80A, a single layer shield formed entirely of the material of layer 80B, and a multi-layer shield 80′ formed of the layers 80A and 80B. Because flux B is related to intensity H according to the equation B=μH, the saturation point (i.e., the point at which magnetic permeability μ is maximized) occurs at the inflection point of the plot of flux vs. magnetic intensity (i.e., the point at which the curve transitions from convex to concave). As shown, the single-layer shield constructed from the material of layer 80A has the highest saturation point HsatA, but its flux B is lower than the other shields over the entire range of magnetic intensities. Conversely, the single-layer shield constructed from the material of layer 80B has the lowest saturation point HsatB, but its flux B is higher than the other shields over the entire range of magnetic intensities. The multi-layer shield 80′ provides a balance between the two single layer shields. Because some of the flux through the multi-layer shield passes through the layer 80B, which has a higher magnetic permeability than layer 80A, the flux through the shield 80′ is increased as compared to the single-layer shield constructed of the material of layer 80A. Moreover, because layer 80A shields layer 80B in the multi-layer shield 80′, the saturation point HsatAB is higher than the saturation point of the single-layer shield constructed of the material of layer 80B. Therefore, the multi-layer shield 80′ can obtain some of the advantages of the higher magnetic permeability of the layer 80B at a fraction of the thickness of a single-layer shield constructed from the material of layer 80B.

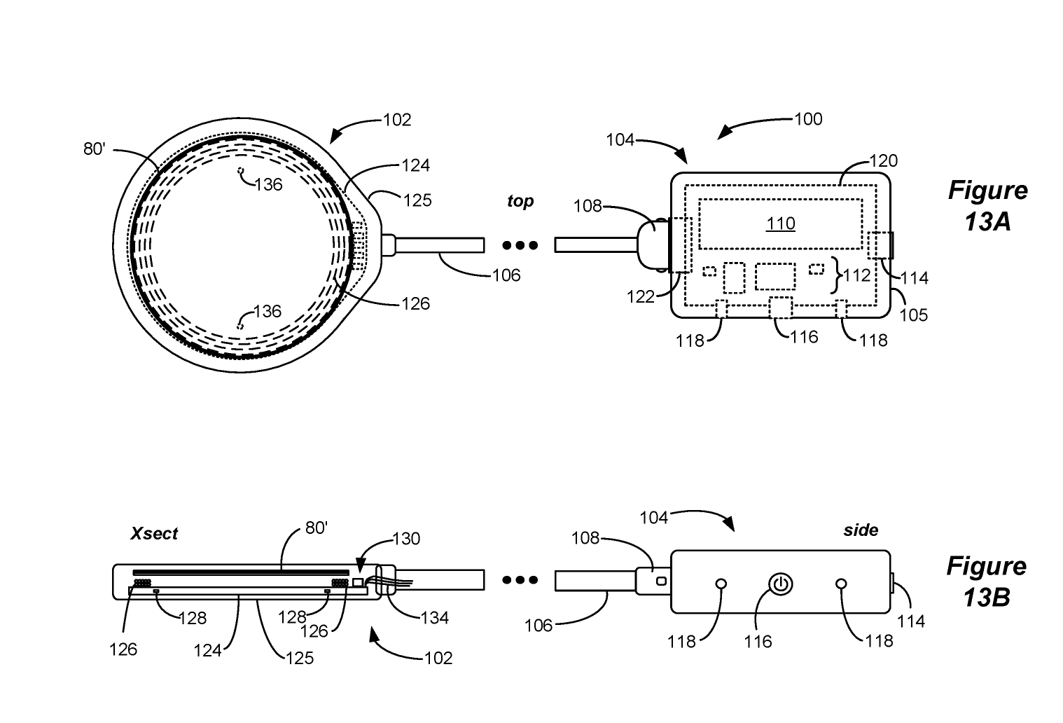

FIG. 12 shows example plots of the inductance of the coil 52 Lcoil over a range of charging currents Icharge for the shields of FIG. 11 and assuming a common thickness of all shields and a common orientation of the coils 52 and 36. As shown, the single-layer shield constructed from the material of layer 80A results in the lowest inductance but the inductance stays stable over the largest range of charging currents. The single-layer shield constructed from the material of layer 80B results in the highest inductance, but the inductance is stable over only a small range of lower values of the charging current. The multi-layer shield 80′ again strikes a balance between the two single-layer shields as it provides an inductance value between those provided by the single-layer shields and is stable over a larger range of charging currents than is the single-layer shield constructed of the material of layer 80B.

While the external charger has to this point been described as a device contained within a single housing, FIG. 13 illustrates a charging system 100 in which an electronics module 104 is separated from a charging coil assembly 102. The electronics module 104 and the charging coil assembly 102 are connected by a cable 106. The cable 106 may be separable from both the electronics module 104 and the charging coil assembly 102 via a port/connector arrangement, but as illustrated cable 106 is permanently affixed to the charging coil assembly 102. The other end of the cable 106 includes a connector 108 that can attach to and detach from a port 122 of the electronics module 104.

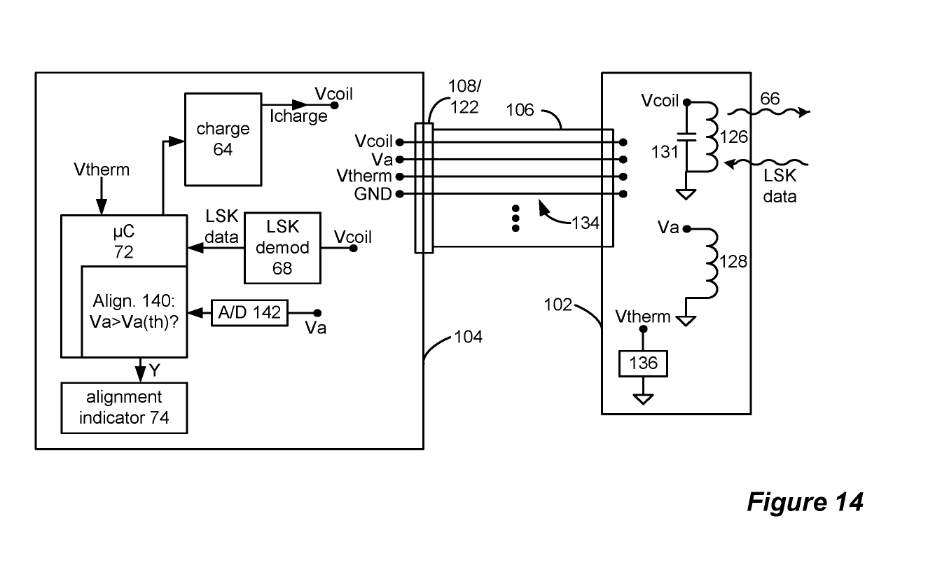

Electronics module 104 preferably includes within its housing 105 a battery 110 and active circuitry 112 needed for charging system operation. Electronics module 104 may further include a port 114 (e.g., a USB port) to allow its battery 110 to be recharged in conventional fashion, and/or to allow data to be read from or programmed into the electronics module, such as new operating software. Housing 105 may also carry a user interface, which as shown in the side view of FIG. 14B can include an on/off switch to begin/terminate generation of the magnetic field 66, and one or more LEDs 118a and 118b. In one example, LED 118a is used to indicate the power status of the electronics module 104. For example, LED 118a may be lit when its battery 110 is charged, and may blink to indicate that the battery 110 needs charging. LED 118b may operate to provide an indication of alignment of the charging coil assembly 102 with the IMD 10. More complicated user interfaces, such as those incorporating a speaker and a display, could also be used. User interface elements can be included on other faces of the electronic module's housing 105, and may be placed such that they are easily viewed for the therapeutic application at hand (e.g., SCS, DBS). Electronics are integrated within the housing 105 of the electronics module 104 by a circuit board 120.

Charging coil assembly 102 preferably contains only passive electronic components that are stimulated or read by active circuitry 112 within the electronics module 104. Such components include the primary charging coil 126, which is mounted above a circuit board 124 that is used to integrate the electronic components within the charging coil assembly 102. The charging coil 126 is energized by charging circuitry 64 (FIG. 14) in the electronics module 104 to create the magnetic charging field 66. The magnetic field 66 generated through energization of the charging coil 126 is directed towards the IMD 10 by the multi-layer shield 80′, which is positioned so that it is located opposite the coil 126 from the patient's tissue when the charging system 100 is in use. In the illustrated embodiment, the shield 80′ is circular and concentric with the coil 126 and has a slightly larger diameter than the coil 126, although the shield 80′ may have a different shape or size than illustrated. In one embodiment, the shield 80′ is suspended above the coil 126 such as by a mechanical support or clamp that affixes the shield 80′ to the housing 125 or other component of the coil assembly 102. Alternatively, the shield may be positioned in a different location. For example, the shield 80′ may be adhered to the coil 126 or to the inside top surface of the housing 125.

Further included within the charging coil assembly 102 are one or more sense coils 128, which as shown in the cross section of FIG. 14B, are preferably formed using one or more traces in the PCB 124. While it is preferred that charging coil 126 comprise a wound conductor, and that the one or more sense coils comprise traces within the circuit board 124, this is not strictly necessary: the charging coil 126 can also be formed from circuit board traces and the one or more sense coils can comprise one or more wound conductors. Note that the charging coil 126 and the one or more sense coils 128 are formed in planes that are parallel, and can also be formed in the same plane. Additional description regarding the one or more sense coils 128 and their use with respect to alignment circuitry 140 can be found in U.S. Patent Application No. 62/350,451, filed Jun. 15, 2016, which is incorporated herein by reference in its entirety.

Further passive components preferably included within the charging coil assembly 102 include one or more tuning capacitors 131, which are utilized to tune the charging coil 126 to its resonant frequency (fres). Each of the one or more sense coils 128 may also be coupled to a tuning capacitor 131, although this is not necessary and is not shown in further circuit diagrams. The charging coil assembly 102 can further include one or more thermistors 136, which can be used to report the temperature of the charging coil assembly 102 to the electronics module 104. Such temperature data can in turn control production of the magnetic field such that the temperature remains within safe limits. See, e.g., U.S. Pat. No. 8,321,029, describing temperature control in an external charging device.

Components in the charging coil assembly 102 are integrated within a housing 125, which may be formed in different ways. In one example, the housing 125 may include top and bottom portions formed of hard plastic that can be screwed, snap fit, ultrasonic welded, or solvent bonded together. Alternatively, housing 125 may include one or more plastic materials that are molded over the electronics components.

Like the external chargers 50 and 50′ described earlier (FIG. 3), the electronics module 104 may include (as part of circuitry 112; FIG. 14) control circuitry 72 that controls charging circuitry 64 to generate the charging current Icharge. This current is passed via connector/port 108/122 through a wire 134 in cable 106 to energize the charging coil 126 to produce the magnetic field 66. The resulting voltage across the charging coil 126, Vcoil, perhaps as dropped in voltage using a voltage divider, can be monitored for LSK communication from the IMD 10 with the assistance of LSK demodulator 68. And again, one or more indications of temperature (Vtherm) can be reported from the one or more thermistors 136 in the charging coil assembly 102 to allow the control circuitry 72 to control production of the magnetic field 66 as mentioned previously. While it is preferable to place control circuitry 72 and other circuitry 112 aspects in the electronics module 104, this is not strictly necessary, and instead such components can reside in the charging coil assembly 102, for example, on its circuit board 124. Thus, electronics module 104 may retain only battery 110 and user interface aspects.

As described above, the multi-layer shield 80′ provides a balance between magnetic permeability and saturation. As such, the multi-layer shield 80′ increases the amount of flux that can be generated through the IMD's charging coil 36 in a way that limits the thickness and associated weight of the shield.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover equivalents that may fall within the spirit and scope of the present invention as defined by the claims.

What is claimed is:

1. An external charger for wirelessly providing power to an implantable medical device, comprising:
a charging coil configured to produce a magnetic field to provide power to the IMD; and
a shield comprising a first ferromagnetic layer and a second ferromagnetic layer, wherein the first ferromagnetic layer is closer to the charging coil than the second ferromagnetic layer, and wherein the first ferromagnetic layer has a higher magnetic saturation point than the second ferromagnetic layer.

2. The external charger of claim 1, wherein the first and second layers have the same thickness.

3. The external charger of claim 2, wherein the thickness of each of the first and second layers is between 1 and 2 mm.

4. The external charger of claim 1, wherein the external charger has a first surface configured to be placed towards a patient's tissue, and wherein the charging coil is positioned between the first surface and the shield.

5. The external charger of claim 1, wherein the charging coil and the shield are concentric.

6. The external charger of claim 1, further comprising a circuit board, wherein the shield is adhered to the circuit board.

7. The external charger of claim 6, further comprising one or more sense coils that are each formed as a trace in the circuit board.

8. The external charger of claim 1, wherein at least one of the first and second ferromagnetic layers comprises a ferrite material.

9. The external charger of claim 8, wherein the ferrite material is scored and held together by a polymer film.

10. The external charger of claim 1, further comprising an electronics module and a charging coil assembly coupled to the electronics module by a cable,
wherein the charging coil and the shield are within the charging coil assembly, and
wherein the electronics module comprises charging circuitry configured to generate a charging current through the charging coil.

11. An external charger for providing power to an implantable medical device (IMD), comprising:
a charging coil configured to generate a magnetic field to provide power to the implantable medical device; and
a shield configured to direct the magnetic field towards the IMD, wherein the shield comprises two or more ferromagnetic layers, wherein each of the layers has a lower magnetic saturation point than any other layer located closer to the charging coil.

12. The external charger of claim 11, wherein each of the layers has a higher magnetic permeability than any other layer located closer to the charging coil.

13. The external charger of claim 11, wherein each of the layers has the same thickness.

14. The external charger of claim 11, wherein the thickness of each layer is between 1 and 2 mm.

15. The external charger of claim 11, wherein the external charger has a first surface configured to be placed towards a patient's tissue, and wherein the charging coil is positioned between the first surface and the shield.

16. The external charger of claim 11, further comprising a circuit board, wherein the shield is adhered to the circuit board.

17. The external charger of claim 16, further comprising one or more sense coils that are each formed as a trace in the circuit board.

18. The external charger of claim 11, wherein at least one of the layers comprises a ferrite material.

19. The external charger of claim 18, wherein the ferrite material is scored and held together by a polymer film.

20. The external charger of claim 11, further comprising an electronics module and a charging coil assembly coupled to the electronics module by a cable,
wherein the charging coil and the shield are within the charging coil assembly, and
wherein the electronics module comprises charging circuitry configured to generate a charging current through the charging coil.
